(19) United States
(12) Patent Application Publication
(10) Pub. No.: US 2025/0073140 A1
(43) Pub. Date: Mar. 6, 2025

(54) ANTIPERSPIRANT COMPOSITIONS CONTAINING A SYNERGISTIC COMBINATION OF COPPER, ALUMINUM, TIN, ZINC, ZIRCONIUM, AND SILVER SALTS, AND, METHODS OF USING

(71) Applicant: Ernest Bloom, Oakland, CA (US)

(72) Inventor: Ernest Bloom, Oakland, CA (US)

(21) Appl. No.: 18/457,284

(22) Filed: Aug. 28, 2023

(51) Int. Cl. A61K 8/26; A61K 8/19; A61K 8/27; A61K 8/28; A61K 8/34; A61K 8/368; A61Q 15/00

(52) U.S. Cl. CPC A61K 8/26; A61K 8/19; A61K 8/27; A61K 8/28; A61K 8/34; A61K 8/345; A61K 8/368; A61Q 15/00; A61K 2800/75

(57) ABSTRACT

Antiperspirant compositions including compositions containing a synergistic (i.e., super-additive) combination of copper, aluminum, tin, zinc, zirconium, and silver salts and methods of using with an efficacy enhancing/irritancy minimizing/skin calming vehicle are described. The solvent may be propylene glycol. The penetration enhancer may be salicylic acid. Methods of using the compositions are also described. Embodiments may be useful in treating excessive perspiration, i.e., hyperhidrosis, (and/or associated undesirable odors) of the axillae, hands, feet, lower back, groin, peri-anal area, buttocks, and other areas.

### CROSS-REFERENCE TO RELATED APPLICATION

This application claims priority to U.S. Provisional Patent Application 63/578,162, Patent Center #62661770, filed Aug. 23, 2023. This application also claims priority to U.S. Provisional Patent Application 61/069,389 filed Mar. 14, 2008, U.S. patent application Ser. No. 12/402,497, Publication number: 20090257970, Filed: Mar. 12, 2009 (initial application not accepted and not appealed, 2012) and European Patent Office Patent Application EP2252252A1. The entire contents of the above documents are incorporated herein by reference.

### TECHNICAL FIELD

This application relates to antiperspirant compositions, and more particularly to antiperspirant compositions containing a synergistic (i.e., super-additive) combination of copper, aluminum, tin, zinc, zirconium, and silver salts and methods of using with an efficacy enhancing/irritancy minimizing/skin calming vehicle.

### BACKGROUND

The conventional (i.e., published in the standard textbooks of dermatology) and widely accepted theory for the mechanism of antiperspirants, involves the formation of aluminum or zirconium hydroxide precipitates, which plug/block the sweat ducts, relatively near the surface of the skin and thus block sweat from reaching the surface of the skin. That is, sweat, made deeper in the skin by the secretory/pumping sweat gland structures, does not reach the skin surface, and the more deeply located secretory/pumping sweat gland cells are inhibited from making sweat due to backpressure related inhibition.

While not wanting to be bound to any particular theory, the present invention was basically designed/invented based on an entirely different proposed theory regarding the mechanism of metal ion antiperspirants.

That theory is as follows:

A) Sweat production involves a multi-unit biochemical pumping complex, which straddles both sides of the membrane of the sweat producing cells in membrane of the skin eccrine and/or other sweat gland cells.
B) This biochemical pumping complex basically functions as a pump and a sodium chloride removing machine/structure, pushing water out of the cell/body, into the lumen of the gland, forming sweat, while removing much, but not all, of the sodium chloride content. (For example, blood sodium concentration=140 mmole/liter (approximately), typical sweat sodium concentration 10-90 mmole/liter (approximately.)) Thus, the biochemical sweat producing complex is essentially sodium chloride removing structure and pump.
C) This biochemical pumping/sodium removing complex is a complicated structure and susceptible to interference/decreased function due to the presence of metal ions binding to and/or altering parts of the complex and stopping full function.
D) Different antiperspirant metal ions are acting at different sites, and, thus would be expected to have a synergistic (i.e., super-additive) effect if combined in a mixture in an antiperspirant formulation.
E) Thus, theoretically, an optimal vehicle for an antiperspirant formulation would be one that promotes deep penetration of the active ingredients into the sweat gland, to reach the actual sweat producing cells. This is in direct contrast to the requirements of accepted conventional theory which requires early precipitation of a metal ion complex nearer the surface to form a plug. Thus, use of salicylic acid with can remove keratin debris (partially) blocking the gland lumen would be useful. Also, the use of a relatively nonirritating penetration enhancing solvent such as propylene glycol would be useful.
F) Other complementary mechanisms are possible, including general damage to the integrity of the cell membrane itself, damage to the function of the smooth muscle cells that pump the sweat inside the lumen of the gland to surface of the skin, death of the entire sweat producing cell, and apoptosis of the entire sweat gland. Also, damage to the lumen itself, of the duct leading to the skin surface can also result in a functional plug, (albeit markedly different from the “plug” described in the conventional theory) which can also cause apoptosis (due to a back pressure-like mechanism) of the sweat producing gland cells deeper in the skin.

As noted above, antiperspirant compositions based on aluminum and aluminum/zirconium are well known and considered the conventional component of topical antiperspirants. In fact, only aluminum and aluminum/zirconium molecules are the only approved active compounds for OTC antiperspirants in the US. (See Over-the-Counter (OTC) Monograph M019: Antiperspirant Drug Products for Over-the-Counter Human Use.)

In terms of patents, for example, Shin et al. (U.S. Pat. No. 4,774,079) describe antiperspirant compositions based on aluminum chlorohydrate, aluminum chloride, and an aluminum zirconium polychlorohydrate complex. Parekh et al. (U.S. Pat. No. 6,902,724) describe an aluminum halide antiperspirant with enhanced efficacy. Carrillo et al. (U.S. Pat. No. 6,991,781) describe an enhanced efficacy antiperspirant based on aluminum-zirconium salts.

Also, relatively recently, in addition to the conventional aluminum/zirconium antiperspirant metal ions, the use of copper has been proposed in a patent application by E. Bloom. (See U.S. Provisional Patent Application 61/069,389 filed Mar. 14, 2008, U.S. patent application Ser. No. 12/402,497, Publication number: 20090257970, Filed: Mar. 12, 2009, and European Patent Office Patent Application EP2252252A1. Patent application(s) were not initially approved and were not appealed.

Furthermore, subsequent to the above patent application, copper chloride has been demonstrated to have antiperspirant activity, in a peer reviewed publication, as described by Bloom, et al, Copper Chloride Antiperspirant Action. Indian Dermatology Online Journal 3(2): p150-151, May August 2012, the entire disclosure of which is hereby incorporated by reference.

Based on the theory of synergistic actions of metal ions, acting on the biochemical pumps that create the sweat (see above), a more effective antiperspirant formulation would be possible by combining multiple appropriate metal ions in a single mixed, formulation, in an appropriate penetration enhancing vehicle.

However, it must be noted that the use of metal ions as antiperspirants, including copper, and aluminum, has, in the past, been hampered by the skin irritancy of these substances. The development of a propylene glycol/salicylic acid vehicle has permitted the use of higher concentrations of aluminum chloride with less irritancy. Described in A. Benohanian et al., International Journal of Dermatology, 37, 701-703 (1998), the entire disclosure of which is hereby incorporated by reference. Also, H. Woolery-Lloyd and W. Valins, J Clin Aesthet Dermatol, 2(6):28-31 (2009) demonstrated that using a propylene glycol/salicylic acid vehicle greatly reduced irritancy (of AlCl₃(H2O)₆), the entire disclosure of which is hereby incorporated by reference.

Also, the use of an improved variation of this propylene glycol/salicylic acid vehicle has permitted the use of copper chloride as an antiperspirant. (See above, Bloom, et al, Copper Chloride Antiperspirant Action.) Without the use of this a novel type of vehicle, i.e., propylene glycol/salicylic acid, the use of copper chloride as an antiperspirant would not be fully practical, compared to using the conventional vehicle of 100% anhydrous ethanol, for example, due to irritancy concerns.

Also, separately, salicylic acid (a penetration enhancer) has been used in certain antiperspirant formulations. For instance, a salicylic acid gel base has been used as a vehicle for aluminum chloride hexahydrate in the treatment of hyperhidrosis, as described in A. Benohanian et al., International Journal of Dermatology, 37, 701-703 (1998, see above.) Salicylic acid has also been used in other antiperspirant compositions based on aluminum and zirconium (e.g., Motley et al., U.S. Pat. No. 5,516,511).

However, despite these advances, even, more effective topical antiperspirant compositions would be useful for those patients suffering from hyperhidrosis (excessive perspiration.) Based the above theory, the formulations would employ a synergistic (i.e., super-additive) combination of appropriate metal ions and a penetration enhancing/irritancy minimizing/skin calming vehicle.

### SUMMARY

This disclosure describes antiperspirant compositions containing a synergistic (i.e., super-additive) combination of copper, aluminum, tin, zinc, zirconium, and silver salts and methods of using, utilizing an efficacy enhancing/irritancy minimizing/skin calming vehicle.

Certain implementations may include the following features. The copper salt can be a copper (II) salt. The copper salt can be copper chloride dihydrate, i.e., CuCl₂(H₂O)₂, or, CuCl₂ (anhydrous.) The copper salt may be dissolved in the composition. In some implementations, the copper salt can be included in the composition in an amount from about 0.1% to about 25% by weight of the total volume of the composition. For example, the copper salt may be included in the composition in an amount from about 1% to about 16% by weight, of the total volume of the composition, e.g., about 2%, about 4%, about 8%, or about 15%.

The other useful metal antiperspirant compounds are: AlCl₃(H2O)₆, SnCl₂(H2O)₂, ZnCl₂, ZrOCl₂(H2O)₃, and, AgCl. These metal compounds, including, CuCl₂(H₂O)₂ act as antiperspirants, in a synergistic manner. These can be incorporated, in an antiperspirant mixture at concentrations from 0.04% to 25%.

Certain implementations may also include the following features. The penetration enhancer may be included in an amount from about 0.1% to about 50% by weight, of the total volume of the composition. For example, the penetration enhancer may be included in an amount from about 1% to about 20%, e.g., from about 1% to about 10%, e.g., about 4%. The composition may include water. For example, water may be included in an amount from about 0.4% to about 10% by volume, of the total volume of the composition. The composition may include an alcohol, e.g., a straight-chain, branched, or cyclic alcohol having from about 1 to about 8 carbon atoms. The alcohol may be ethyl alcohol. The alcohol may be present in an amount from about 0.1% to about 50% by volume, of the total volume of the solution, e.g., from about 1% to about 40%, e.g., about 20%.

In implementations, the composition may also include an organic carrier. The organic carrier may be selected from the group consisting of straight-chain, branched, and cyclic alcohols, polyols (including diols and glycols), esters, and carbonates having from about 1 to about 6 carbon atoms, and combinations thereof. The organic carrier can be propylene glycol.

Implementations include methods for reducing or preventing perspiration and for treating hyperhidrosis. Implementations also include methods for treating or preventing undesirable odors associated with human sweat glands and skin. Certain methods comprise applying an effective amount of a composition containing a copper salt and a penetration enhancer to the skin of a human subject. In some methods, the composition may also include an aluminum or zirconium salt (or a mixture thereof). Other methods include first applying an effective amount of a composition containing a copper salt and a penetration enhancer followed by applying an effective amount of a composition containing an aluminum or other metal salt (or a mixture thereof, listed above). In some methods, the subject may suffer from social sweating associated with normal daily activities.

As noted above, certain implementations may have one or more of the following advantages. The compositions can combine in a synergistic (i.e., super-additive) manner. The compositions are effective in reducing perspiration and in treating excessive perspiration or hyperhidrosis, particularly with, but not limited to, subjects not responding well to other conventional treatments. The compositions may reduce perspiration for an extended time after application so that application may be necessary only a few times per week, or month, to achieve and maintain the desired degree of reduced perspiration. The compositions may be effective for treating the axillae, hands, feet, lower back, groin, peri-anal area, buttocks, and other areas.

The optimally effective combination will include a combination of multiple metal salts for a synergistic (i.e., greater than the sum of their separate effects) efficacy as an antiperspirant. The application utilizes/comprises a synergistic combination of copper, aluminum, tin, zinc, zirconium (zyrconyl) and silver salts and methods of using, utilizing an efficacy enhancing/irritancy minimizing/skin calming vehicle.

The details of four implementations are set forth in the description and examples below. Other features, objects, and advantages will be apparent from the description, examples, and the claims.

### DETAILED DESCRIPTION

This disclosure describes antiperspirant compositions comprising a copper salt, and other metals (noted above) and an efficacy enhancing/irritancy minimizing/skin calming/sweat gland penetration enhancing vehicle. The term “dissolved” as used herein means that the copper is substantially dissolved in and/or colloidally dispersed in the composition. Concentrations of constituents are by weight of total volume of the composition (weight/volume) for solid constituents such as metal salts, metal salt hydrates, and salicylic acid, and are by volume of the total volume of the composition (volume/volume) for liquid constituents such as water and alcohol. In each case, concentrations are initial concentrations, i.e., those at the time the composition is made (e.g., shortly after the constituent is added to the composition). It is understood, and within the scope of this invention, that concentrations of certain constituents at later times may be different than the initial concentrations (e.g., due to evaporation).

As used herein, the term “copper salt” includes copper ions in the form of, or derived from, one or more copper salts, including copper salt hydrates. The copper salts used herein are chiefly salts or salt hydrates of copper (II).

Examples of copper salts useful herein include, without limitation, chlorides, bromides, iodides, sulfates, nitrates, cyanides, acetates, acetylacetates, oxalates, carbonates, formates, oleates, and maleates, or hydrates thereof, (for example, but not limited to, copper(II) chloride dihydrate (CuCl₂(H₂O)₂) and copper(II) sulfate pentahydrate (Cu(SO3)(H2O)5)). Other inorganic and organic salts may also be used, such as those found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, PA (2005) at p. 732, Table 38-5.

While not wanting to be bound to any particular theory, it is thought that the antiperspirant effect of the copper salts, and other metal salts, described herein is chiefly due to the therapeutic inhibition of cell metabolic, sweat producing, activity by the metal or metal ion, located primarily in the membrane of the sweat producing gland cells, as noted above. Target cells of this inhibition include cells that are part of, and related to, the sweat glands and associated structures. As noted above, this proposed mechanism is different from the conventional theory/mechanism of aluminum and aluminum/zirconium salts. A significant portion of the inhibitory biological action of copper, and other metal compound, is thought to come from its redox properties, on the biochemical sweat pump structure. Copper, for example, can catalyze the production of highly reactive radical ions such as the hydroxyl radical. An increase in free radicals of this type is known as oxidative stress, and is an active area of research in a variety of diseases where copper may play an important role. For instance, oxidative stress can inhibit cell growth or cause cell death, as reported, for example, by L. M. Gaetke and C. K. Chow, Toxicology, 189(1-2), 147-63 (2003); and G. Filomeni et al., J. Biol. Chem., 282(16), 12010-21 (2007). Copper may interfere with other vital cellular functions, including highly energy-dependent membrane pumping functions, which occur primarily in the membranes of the cells in the sweat glands. It is therefore thought that copper (e.g., Cu(II) and Cu(I)) may damage, disrupt, or even cause apoptosis or cell death of some the sweat gland cells, thereby reducing or eliminating sweat production.

In addition to copper, aluminum, tin, zinc, zirconium, and silver salts also inhibit the biochemical sweat producing cell units. Each metal salt is thought to be acting via different target sites in the cell membrane sweat pumping units making it more difficult for the cell to overcome the damage to its sweat producing biochemical unit, thus producing a synergistic (i.e., greater the sum of their separate effects) action.

As used herein, the term “penetration enhancer” means an agent which enables or increases entrance of substances primarily into the lumen of the sweat gland duct and penetration down to the active sweat pumping cells. A penetration enhancer may also increase percutaneous absorption through the epidermis, to the dermis. Penetration enhancers of the disclosed compositions include salicylic acid and urea. Other penetration enhancers, such as dimethyl sulfoxide (DMSO) may also be used. Other suitable penetration enhancers such as those listed in Remington (2006), at page 959 (see Table 47-9), may also be used, including surfactants such as sodium lauryl sulfate, sodium laurate, sodium dodecylsulfate, polyoxyethylene-20-cetylether, and polyoxyethylene-9-lauryl ether, bile salts and derivatives such as sodium glycocholate, fatty acids and derivatives such as oleic acid and caprylic acid, and other agents such as citric acid, EDTA, decyl methyl sulfoxide, glycerol, azone, and cyclodextrin.

As described herein, salicylic acid and urea may act as penetration enhancers of the active antiperspirant ingredient. Salicylic acid is a keratolytic agent, causing cells of the epidermis to slough off, and causing keratin-lined skin pores and ducts to remain more patent/open. Salicylic acid is known in the treatment of eczema, keratosis pilaris, psoriasis, and related skin conditions. While not wanting to be bound to any particular theory, it is thought that salicylic acid may enhance biological access or absorption of the active antiperspirant ingredient, as disclosed in A. Benohanian et al., above, by improving access to and/or maintaining enhanced opening (patency) of the sweat gland ducts. It is thought that urea may have a similar effect since urea is also a widely used keratolytic agent for treating the skin conditions mentioned above. Salicylic acid may also have other beneficial properties, such as promoting normal skin (non-perspiration related) hydration levels, as also disclosed in A. Benohanian et al., above.

Certain implementations include an organic carrier or vehicle. A suitable organic carrier will have certain desirable properties including the ability to (i) dissolve or disperse the metal salt(s) and the penetration enhancer; (ii) enable a stable, homogeneous composition to be formed; (iii) leave little to no residue after application. Suitable organic carriers may also be selected for viscosity, lubricity, and other properties. Suitable organic carriers used herein include straight-chain, branched, and cyclic alcohols, polyols (including diols and glycols), esters, and carbonates having from about 1 to about 6 carbon atoms. Examples include, without limitation, methanol, ethanol, n-propanol, isopropanol, n-butanol, 2-methoxyethanol, 2-ethoxyethanol, ethylene glycol, 1,2-propylene glycol, diethylene glycol, isopropanol, isobutanol, diethylene glycol monoethylether, 1,3-butylene glycol, 2,3-butylene glycol, dipropylene glycol, and 2,4-dihydroxy-2-methylpentane. Other suitable organic carriers (including excipients and colloidal systems) may also be used, such as those listed in B. J. Bowman et al., Colloidal Dispersions, Chap. 21, in Remington (2006); and M. M Crowley, Solutions, Emulsions, Suspensions, and Extracts, Chap. 39 in Remington (2006). For clarity, certain organic carriers may also act as penetration enhancers.

In various implementations, water, an alcohol, or both water and an alcohol may be added to the composition to alter the solubility, viscosity, lubricity, and other properties of the composition. Suitable alcohols include straight-chain, branched, and cyclic alcohols having from about 1 to about 8 carbon atoms. Exemplary alcohols include, without limitation, methanol, ethanol, isopropanol, n-propanol, n-butanol, isobutanol, tert-butyl alcohol, 1-hexanol, 1-octanol, and cyclohexanol. Other suitable alcohols may also be used.

Methods of using the compositions disclosed herein are also described. In some implementations, methods of reducing or preventing perspiration, of treating hyperhidrosis, and of treating or preventing undesirable odors associated with human sweat glands and skin include applying to the skin of a human an effective amount of a composition comprising multiple metal salts and an efficacy enhancing/irritancy minimizing/skin calming/sweat gland penetration enhancing vehicle

In some implementations, the subject may suffer from social sweating associated with normal daily activities. In some implementations, the subject may suffer from exercise-induced sweating. The compositions described herein will typically be administered locally and topically at the desired location, for example, but not limited to, the axillae, hands, feet, and groin areas. The compositions may be administered by direct application or with a suitable skin applicator. The composition may also be administered via or as part of any other suitable vehicle, applicator, or deodorant or antiperspirant formulation, including, without limitation, a dropper (e.g. an eyedropper or pipette), a sponge-top type applicator, a spray, aerosol, roll-on, stick, gel, lotion, cream, salve, or powder.

In accordance with the present invention, there may be employed conventional dermatology, pharmacology, and chemistry techniques within the skill of the art. The invention is further described in the following examples, which do not limit the scope of the claimed invention.

### EXAMPLES

### Example 1: Preparation of Solutions (all Mixed and Dissolved in Propylene Glycol, See Below)

| | (final concentrations) | |
|---|---|---|
| Formulation A | | |
| Ethanol (anhydrous) | 4% | v/v |
| Salicylic Acid | 4% | w/v |
| CuCl₂(H₂O)₂ | 4% | w/v |
| AlCl₃(H2O)₆ | 1% | w/v |
| SnCl₂(H2O)₂ | 0.4% | w/v |
| ZnCl₂ | 0.4% | w/v |
| ZrOCl₂(H2O)₈ | 0.04% | w/v |
| AgCl | 0.04% | w/v |
| Formulation B | | |
| Ethanol (anhydrous) | 4% | v/v |
| Salicylic Acid | 4% | w/v |
| CuCl₂(H₂O)₂ | 4% | w/v |
| AlCl₃(H2O)₆ | 0 | |
| SnCl₂(H2O)₂ | 0.4% | w/v |
| ZnCl₂ | 0.4% | w/v |
| ZrOCl₂(H2O)₈ | 0.04% | w/v |
| AgCl | 0.04% | w/v |
| Formulation C | | |
| Ethanol (anhydrous) | 4% | v/v |
| Salicylic Acid | 4% | w/v |
| CuCl₂(H₂O)₂ | 4% | w/v |
| AlCl₃(H2O)₆ | 0 | |
| SnCl₂(H2O)₂ | 0.4% | w/v |
| ZnCl₂ | 0.4% | w/v |
| ZrOCl₂(H2O)₈ | 0 | |
| AgCl | 0.04% | w/v |
| Formulation D | | |
| Ethanol (anhydrous) | 4% | v/v |
| Salicylic Acid | 4% | w/v |
| CuCl₂(H₂O)₂ | 4% | w/v |
| AlCl₃(H2O)₆ | 1% | w/v |
| SnCl₂(H2O)₂ | 0.4% | w/v |
| ZnCl₂ | 0.4% | w/v |
| ZrOCl₂(H2O)₈ | 0 | |
| AgCl | 0.04% | w/v |

For example, to make 1000 ml of Formulation A, a sufficient amount (typically, about 400 ml depending, in part, on the amount of other constituents being added) of propylene glycol is added to a measuring beaker. While stirring at room temperature, the following ingredients are added (for Formulation A, as noted above):
40 ml ethanol (anhydrous) (final concentration 4% v/v)
40 gr salicylic acid (final concentration 4% w/v)
40 gr CuCl₂(H₂O)₂ (final concentration 4% w/v)
10 gr AlCl₃(H2O)₆ (final concentration 1% w/v)
4 gr SnCl₂(H2O)₂ (final concentration 0.4% w/v)
4 gr ZnCl₂ (final concentration 0.4% w/v)
0.4 gr ZrOCl₂(H2O)₃ (final concentration 0.04% w/v)
0.4 gr AgCl (final concentration 0.04% w/v)

Additional propylene glycol is then added until the total volume was 1000 ml. Any undissolved metal pellets/clumps are crushed, and the solution was allowed to stir at room temperature until all of the salicylic acid/metal salts are substantially dissolved (typically for about 24 hours, or longer). The solution is stored in a glass bottle at room temperature. The preparation of formulations B, C, and, D are done in an analogous manner.

### Example 2: Treatment of Axillae

A human male subject was identified as sweating associated with athletic activity. Approximately 1-3 mls of the solution (Example C) was applied at each treatment to the axillae of the subject, either directly or using an applicator. The subject treated himself at bedtime. Within two successive daily bedtime applications, sweat production was greatly decreased. Once achieved, this antiperspirant effect could be maintained by similar reapplications approximately once per week.

### Example 3: Formulations Demonstrating Synergy of Action (Copper and Aluminum)

Four human male volunteers aged, 54-69 years old, were recruited. Solutions containing ethanol (anhydrous), salicylic acid, 0-4% CuCl₂(H₂O)₂, and 0-4% AlCl₃(H2O)₆ and other compounds (see below) were prepared. All formulations (see below) were dissolved in propylene glycol.
I) 4% AlCl₃(H2O)₆
   0% CuCl₂(H₂O)₂
and 4% Salicylic Acid, 10% Ethanol: 20%, Water: 0.4% %
II) 0% AlCl₃(H2O)₆
   4% CuCl₂(H₂O)₂
and 4% Salicylic Acid, 10% Ethanol: 20%, Water: 0.4% %
III) 4% AlCl₃(H2O)₆
   4% CuCl₂(H₂O)₂
and 4% Salicylic Acid, 10% Ethanol: 20%, Water: 0.4% %
IV) 1% AlCl₃(H2O)₆
   4% CuCl₂(H₂O)₂
and 4% Ethanol (anhydrous), 4% Salicylic Acid, 0.4% SnCl₂(H2O)₂,
0.4% ZnCl₂, 0.04% ZrOCl₂(H2O)₈, 0.04%, AgCl
V) 0% AlCl₃(H2O)₆
   4% CuCl₂(H₂O)₂
and 4% Ethanol (anhydrous), 4% Salicylic Acid, 0.4% SnCl₂(H2O)₂,
0.4% ZnCl₂, 0.04% ZrOCl₂(H2O)₈, 0.04%, AgCl
VI) 1% AlCl₃(H2O)₆
   4% CuCl₂(H₂O)₂
and 4% Ethanol (anhydrous), 4% Salicylic Acid, 0.4% SnCl₂(H2O)₂,
0.4% ZnCl₂, 0% ZrOCl₂(H2O)₈, 0.04%, AgCl
VII) 0% AlCl₃(H2O)₆
   4% CuCl₂(H₂O)₂
and 4% Ethanol (anhydrous), 4% Salicylic Acid, 0.4% SnCl₂(H2O)₂,
0.4% ZnCl₂, 0% ZrOCl₂(H2O)₈, 0.04%, AgCl

The volunteer patients applied the solutions (either I, II, III, IV, V, VI, or VII) to the left axilla or to the lower back region. over a one day to two-week period.

The degree of perspiration, for the axillary part of the study, was determined from the diameter of sweat rings on cotton shirts worn by the subjects after exercise. For the lower back part of the study, results were recorded from the subjective impression/result recorded by the subject.

The results indicated that the sweat reducing activity of the solution containing both copper and aluminum metal ions were the most effective at reducing sweating. The results implied synergy (i.e., super-additive) action/activity in reducing sweating.

What is claimed is:

1. An antiperspirant composition containing a synergistic combination of copper, aluminum, tin, zinc, zirconium and silver salts and dissolved/suspended in an efficacy enhancing/irritancy minimizing/skin calming vehicle.

2. The composition/formulation: Ethanol (anhydrous) 4% v/v, Salicylic Acid 4% w/v, CuCl₂(H₂O)₂4% w/v, AlCl₃(H2O)₆1% w/v, SnCl₂(H2O)₂ 0.4% w/v, ZnCI₂ 0.4% w/v, ZrOCl₂(H2O)₈ 0.04% w/v, AgCl 0.04% w/v.

3. The composition/formulation: Ethanol (anhydrous) 4% v/v, Salicylic Acid 4% w/v, CuCl₂(H₂O)₂4% w/v, AlCl₃(H2O)₆0% w/v, SnC1₂(H2O)₂ 0.4% w/v, ZnCl₂ 0.4% w/v, ZrOCl₂(H2O)₃ 0.04% w/v, AgCl 0.04% w/v.

4. The composition of claims 2 and 3 wherein the organic carrier is selected from the group consisting of straight-chain, branched, and cyclic alcohols, polyols, esters, and carbonates having from about 1 to about 6 carbon atoms, and combination thereof.

5. The composition of claims 2 and 3 wherein the organic carrier is propylene glycol.

6. Antiperspirant formulations containing practical different concentrations of the following metal salts: copper, aluminum, tin, zinc, zirconium, and silver, at concentrations from 0 to 25%.

7. Antiperspirant formulations in claims 2 and 3 used as deodorants for treating or preventing undesirable odors associated with human sweat.

8. In addition to treating the axillae for perspiration and/or odor control, the formulations may be used to treat hands, feet, lower back, groin, peri-anal area, buttocks, and other areas.

9. An article of manufacture comprising a composition of any of claims 1-8.
